(19) United States
(12) Patent Application Publication
(10) Pub. No.: US 2014/0149016 A1
(43) Pub. Date: May 29, 2014

(54) Conduit Degradation Detection System and Method

(71) Applicant: CATERPILLAR INC., Peoria, IL (US)

(72) Inventor: Amiyo Basu, Peoria, IL (US)

(73) Assignee: CATERPILLAR INC., Peoria, IL (US)

(21) Appl. No.: 13/688,944

(22) Filed: Nov. 29, 2012

(51) Int. Cl. G01N 33/00; G01L 1/00

(52) U.S. Cl. CPC G01N 33/00; G01L 1/00
USPC 701/102; 73/865.8; 73/760

(57) ABSTRACT

A flexible conduit may include a first layer defining an inner surface, a second layer defining an outer surface, and a metal reinforcement element disposed between the first and second layers, the metal reinforcement element having associated therewith a first physical property. One or more sensors is coupled to the metal reinforcement element and configured to detect the first physical property and generate a signal indicative of the first physical property. A controller is operably coupled to the sensor and configured to receive the signal and display physical property data, thereby to determine potential degradation of the conduit.

### TECHNICAL FIELD

The present disclosure generally relates to systems and methods for detecting degradation in conduits, and more particularly to systems and methods for detecting changes in the physical characteristics of a conduit.

### BACKGROUND

Conduits are generally known for communicating various types of fluid in numerous industrial applications. For example, the conduit may communicate a transmission fluid, a brake fluid, fuel, engine oil, or other types of fluid that may be used by an engine for construction, automotive, aerospace, chemical processes, or other types of applications. The fluid pressure in the conduit may be substantially steady-state or may fluctuate based on operation of the system in which the conduit is used.

In one example, the conduit may be a high pressure reinforced hose used to communicate hydraulic fluid used in fluid power operated machines. The high pressure hose may provide a flexible connection between moving parts of a hydraulic circuit provided on the machine. In general, such hoses may include a hollow polymeric inner tube on which successive cylindrical layers of reinforcing material, such as wire or textile, may be concentrically applied to withstand the radial and axial forces generated by the high pressure fluid within the inner tube.

Depending on the application, the high pressure reinforced hose may encounter various types of forces during use. For example, some hoses may require a hose construction having both high burst strength and long-term fatigue resistance. Using conventional technology, the burst strength of a hose design may be increased by adding additional reinforcing material and/or layers, which may negatively impact the flexibility of the hose. Alternatively, the tensile strength of each layer of reinforcement material may be increased, which may reduce the ability of the hose to resist fatigue.

Regardless of the design, high pressure reinforced hoses may fail during use. In machines that are continuously operated often at a steady state, such as mining trucks, the hose failure is typically due to insufficient burst strength. In other machines that experience more variable operation and therefore potentially rapid cycles of fluid pressure, such as excavators, the hose failure is often due to insufficient fatigue resistance. While the hose itself may be a relatively inexpensive part, hose failure may damage other more costly components of the machine, and therefore significant repair costs may be incurred. Additionally, the operator may lose revenue due to machine downtime. Accordingly, it may be advantageous to quickly detect hose failure to minimize costs.

Some systems have been proposed to identify when leaks occur in a hose. U.S. Patent Application Publication 2010/0174495 to Pereira et al., for example, discloses a degradation detection system for a hose assembly that incorporates conductive layers separated by a non-conductive layer and disposed within a hose body. An electrical characteristic, such as capacitance, between the conductive layers is monitored, and a warning is generated when the electrical characteristic exceeds a predetermined threshold. Accordingly, the Pereira system does not measure hose degradation per se, but instead infers hose degradation by a change in the electrical characteristic, as may occur in the presence of a fluid leak.

### SUMMARY

In accordance with one aspect of the disclosure, a system is provided for detecting degradation of a conduit that includes a conduit body defining a conduit inner surface and a conduit outer surface, and a reinforcement element disposed in the conduit body and having a first physical property. A sensor is operably coupled to the reinforcement element and configured to detect the first physical property and generate a first physical property signal indicative of the first physical property. A controller is operably coupled to the sensor and configured to receive the first physical property signal.

In another aspect of the disclosure that may be combined with any of these aspects, a system is provided for detecting degradation of a flexible conduit. The system includes a conduit assembly having a first polymeric conduit layer defining a conduit inner surface, a second polymeric conduit layer defining a conduit outer surface, and a metal reinforcement element disposed between the first and second polymeric conduit layers, the metal reinforcement element having associated therewith a hoop stress, an axial stress, and a shear stress. An array of strain gages is operably coupled to the metal reinforcement element, each strain gage in the array of strain gages being configured to continuously detect the hoop stress, the axial stress, and the shear stress and generate a structural deformation signal indicative of the hoop stress, the axial stress, and the shear stress. A controller is operably coupled to the array of strain gages and configured to receive each of the physical property signals and display physical property data based on the physical property signals.

In another aspect of the disclosure that may be combined with any of these aspects, a method is provided of detecting degradation of a conduit having a conduit body and a reinforcement element disposed in the conduit body. The method includes determining a first physical property associated with the reinforcement element, the first physical property including at least one property selected from a group of physical properties including hoop stress, axial stress, and shear stress, communicating the first physical property to a controller, and determining a fault condition in response to the first physical property meeting a fault threshold.

### BRIEF DESCRIPTION OF THE DRAWINGS

FIG. 1 is a perspective view, partially cut away, illustrating an exemplary hose assembly having a conduit degradation detection system according to the present disclosure.

FIG. 2 is an enlarged plan view of a portion of the hose assembly of FIG. 1 illustrating a strain gage orientation on a reinforcing element.

### DETAILED DESCRIPTION

Embodiments of conduit degradation detection systems and methods are disclosed for use in high pressure fluid applications, such as hydraulic circuits used on machines. In the exemplary embodiments described herein, the conduit may be a high pressure hose having a reinforcement element. A sensor may be operably coupled to the reinforcement element and configured to determine a physical property associated with the reinforcement element. For example, the sensor may be a strain gage that determines a hoop stress, an axial stress, a shear stress, or other property. Information regarding the physical property may be communicated to a controller, which in turn may determine a fault condition should the physical property meet a threshold value. Accordingly, the systems and methods disclosed herein provide a more direct indication of the actual forces to which the conduit has been subjected, thereby permitting an early diagnosis of potential conduit failure prior to the presence of a leak.

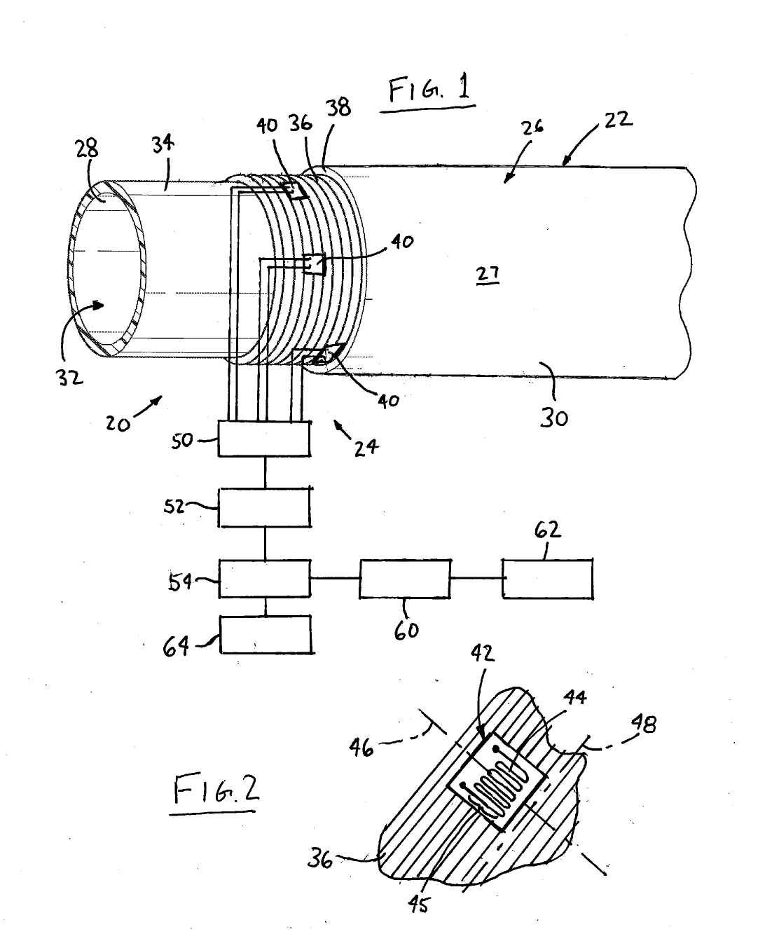

Referring to the drawings, FIG. 1 illustrates one embodiment of a conduit degradation detection system, generally designated at 20. The conduit degradation detection system 20 may include a conduit assembly, such as hose assembly 22, and a degradation detector 24 operably coupled to the hose assembly 22.

The hose assembly 22 includes a hose 26 having a hose body 27 with a hose inner surface 28 and a hose outer surface 30. The hose inner surface 28 defines a passageway 32 through which a fluid may pass. In the illustrated embodiment, the hose 26 has a multi-layer construction that permits the hose to be flexible while providing sufficient strength to withstand the fluid pressure without leaking. For example, the hose 26 may include a first hose layer 34 that defines the hose inner surface 28. A reinforcement element may be disposed around the first hose layer 34. In the illustrated embodiment, the reinforcement element is a wire 36 that is wound in a spiral configuration around the first hose layer 34. A second hose layer 38 extends around the wire 36 and defines the hose outer surface 30.

The first and second hose layers 34, 38 may be formed of a polymer material, such as rubber or plastic, or another material depending on the requirements of the particular application. The wire 36 may be formed of a material that is relatively more rigid than the material used for the first and second hose layers 34, 38, such as a metal. The wire 36 may extend the entire length and span the entire circumference of the hose body 27.

The degradation detector 24 is schematically shown in FIG. 1 as including one or more sensors 40 for determining a physical property of the hose 26 and generating a signal indicative of that physical property. Each sensor 40 is operably coupled to the hose body 27 to permit monitoring of the physical property. While the illustrated embodiment shows three sensors 40 coupled to the hose assembly 22, it will be appreciated that a greater or lesser number of sensors 40 may be used, depending on the application. Furthermore, while the sensors 40 are shown in FIG. 1 as being located at substantially the same axial point along the hose 26, the sensors 40 may be spaced along the entire axial length of the hose 26 as needed.

As best shown in FIG. 2, the sensors 40 may be configured as strain gages 42 that are mechanically attached to the wire 36 forming the reinforcement element, and therefore are configured to determine a physical property associated with the wire 36. More specifically, the strain gages 42 may determine structural deformation of the hose 26, since the wire 36 will deform with the hose body 27. Each strain gage 42 may be oriented to determine different types of deformation of the wire 36. As shown in FIG. 2, for example, the strain gage 42 may have a gage grid 44 with an active length 45 disposed along a gage axis 46. The strain gage 42 may be oriented so that the gage axis 46 is substantially perpendicular (i.e., within approximately 10 degrees) to a wire axis 48 defined by the spiral configuration of the wire 36. When oriented in this manner, the feedback provided by the strain gage 42 may be used to determine various types of physical properties of the wire 36 that are indicative of structural deformation of the hose 26, such as hoop stress, axial stress, and shear stress.

Returning to FIG. 1, the degradation detector 24 may further include a sensor circuit 50 configured to measure the signal provided by the sensor 40. When the strain gage 42 is used as the sensor 40, the sensor circuit 50 may include a strain gage circuit such as a full, half, or quarter Wheatstone bridge configured to measure the fine changes in the signal output provided by the strain gage 42. The sensor circuit 50 may further include a power source for providing DC power to the sensor circuit 50 and sensor 40. The power source may provide continuous power to the sensor circuit 50 and strain gage 42, thereby to provide continuous monitoring of the physical property associated with the hose assembly 22.

Still further, the degradation detector 24 may include a signal conditioner 52 for converting the sensor signal into a more readily usable format. The signal conditioner 52 may include one or more amplifiers, filters, offset nullifiers (either hardware- or software-based), calibrators, or other components for conditioning the signal provided by the sensor 40. During conditioning, the signal may be converted from an analog signal to a digital signal.

The conduit degradation detection system 20 may further include a controller 54 communicatively coupled to the degradation detector 24 and configured to process the signal provided by the sensor 40. The controller 54 may communicate with the degradation detector 24 via wired or wireless connections. The controller 54 may be a digital computer, microcontroller, processor, or other type of control component with associated memory and timing circuitry.

### INDUSTRIAL APPLICABILITY

The present disclosure is applicable to conduits used to communicate high pressure fluid, such as hoses used in hydraulic circuits provided on machines. The conduit degradation detection system 20 may include one or more sensors 40 that directly determine changes in one or more physical properties of the hose 26, such as structural deformation. The sensors 40 provide a signal indicative of the physical property that may be interpreted to identify hose degradation and/or weakness prior to failure of the hose. In some embodiments, the signal may be used to modify machine operation to prolong hose integrity until the machine can be serviced.

More specifically, the controller 54 may include a processing unit having an algorithm configured to determine the presence of a fault condition, indicative of hose degradation and/or a leak, based on the magnitude of the physical property determined by the sensor 40. The algorithm may continuously or periodically read the physical property value and compare it to a fault threshold. When the fault threshold is met, the algorithm may generate a fault signal. The fault threshold may be a predetermined value that serves as a limit for the physical property of the hose assembly 22. Alternatively, the fault threshold may be determined based on observed parameters during operation of the hose assembly 22. Still further, the fault threshold may be a range of values that provide upper and lower limits for the physical property.

The fault threshold may be particularly adapted for the manner in which the hose assembly 22 will be used. In some applications, the hose assembly 22 will experience continuous operation where the fluid pressure is substantially constant. For such steady state operation, the fault threshold may be more precise and/or constant. In other applications, the hose assembly 22 may experience intermittent or transient fluid pressure. When used in such cyclical pressure applications, the fault threshold may also shift to accommodate changes in operating parameters.

The algorithm may also consider other parameters in addition to the physical property of the hose assembly when determining a fault condition. For example, the hose assembly 22 may be used on a machine having an electronic control module (ECM) 60 operably coupled to an engine 62. The ECM 60 may receive information regarding machine operating parameters, such as engine temperature or engine speed, that may be used in combination with the physical property of the hose assembly to determine when a fault condition is present. For example, a fault condition may be determined when the sensors 40 indicate an increase in structural deformation of the hose 26 and the ECM 60 indicates a relatively constant engine temperature, as would be expected during steady state operation of the machine. In this case, the fluid pressure should be relatively constant and therefore the increased structural deformation may be due to degradation of the hose instead of a change in machine operation. Alternatively, when the sensors 40 indicate an increase in structural deformation but the ECM 60 also indicates an increase in engine temperature, as would be expected during transient operation of the machine, then the hose 26 may be responding to an increase in fluid pressure and therefore a fault condition may not be present.

The algorithm may be further configured to take certain actions in response to the determination of a fault condition. For example, an alert signal may be communicated to a display 64 to notify the operator of potential hose degradation and/or failure. Additionally or alternatively, the algorithm may include a feed forward loop that communicates an override signal to the ECM 60. The override signal may command the machine to operate in a manner that reduces the fluid pressure through the hose 26, thereby preserving hose integrity until it can be serviced. In some embodiments, the controller 54 and algorithm may be incorporated into the ECM 60, in which case the override signal is communicated directly to the engine 62. In other embodiments that have a dedicated controller 54, the override signal may be communicated to the ECM 60, which in turn will communicate with the engine 62 to modify operation of the machine.

It will be appreciated that the foregoing description provides examples of the disclosed assembly and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A system for detecting degradation of a conduit, comprising:
a conduit body defining a conduit inner surface and a conduit outer surface;
a reinforcement element disposed in the conduit body and having a first physical property;
a sensor operably coupled to the reinforcement element and configured to detect the first physical property and generate a first physical property signal indicative of the first physical property; and
a controller operably coupled to the sensor and configured to receive the first physical property signal.

2. The system of claim 1, in which the first physical property comprises a structural deformation.

3. The system of claim 2 in which the conduit body comprises a first conduit layer defining the conduit inner surface and a second conduit layer defining the conduit outer surface, and in which the reinforcement element is disposed between the first and second conduit layers.

4. The system of claim 3, in which the first and second conduit layers are formed of polymer material.

5. The system of claim 4, in which the reinforcement element comprises a metal wire configured in a spiral pattern around the conduit inner surface.

6. The system of claim 5, in which the sensor comprises a strain gage.

7. The system of claim 6, in which the strain gage has an active length oriented along a gage axis, the metal wire defines a wire axis, and the strain gage is oriented so that the gage axis is substantially perpendicular to the wire axis.

8. The system of claim 7, in which the first physical property includes at least one property selected from a group of physical properties including hoop stress, axial stress, and shear stress.

9. The system of claim 1, in which the sensor is configured to continuously detect the first physical property.

10. The system of claim 1, in which the controller is configured to determine a fault condition in response to the first physical property signal meeting a fault threshold.

11. The system of claim 10, in which the conduit conducts hydraulic fluid on a machine, and in which the controller comprises an electronic control module provided on the machine.

12. The system of claim 11, in which the electronic control module is operably coupled to an engine, and in which the electronic control module is further configured to communicate an override signal to the engine in response to the fault condition.

13. A system for detecting degradation of a flexible conduit, comprising:
a conduit assembly including:
a first polymeric conduit layer defining a conduit inner surface;
a second polymeric conduit layer defining a conduit outer surface; and
a metal reinforcement element disposed between the first and second polymeric conduit layers, the metal reinforcement element having associated therewith a hoop stress, an axial stress, and a shear stress;
an array of strain gages operably coupled to the metal reinforcement element, each strain gage in the array of strain gages being configured to continuously detect the hoop stress, the axial stress, and the shear stress and generate a structural deformation signal indicative of the hoop stress, the axial stress, and the shear stress; and
a controller operably coupled to the array of strain gages and configured to receive each of the physical property signals and display physical property data based on the physical property signals.

14. The system of claim 13, in which the controller is configured to determine a fault condition in response to the structural deformation signal meeting a fault threshold.

15. The system of claim 14, in which the conduit conducts hydraulic fluid on a machine, and in which the controller comprises an electronic control module provided on the machine, wherein the electronic control module is operably coupled to an engine and further configured to communicate an override signal to the engine in response to the fault condition.

16. A method of detecting degradation of a conduit having a conduit body and a reinforcement element disposed in the conduit body, the method comprising:
determining a first physical property associated with the reinforcement element, the first physical property including at least one property selected from a group of physical properties including hoop stress, axial stress, and shear stress;
communicating the first physical property to a controller; and
determining a fault condition in response to the first physical property meeting a fault threshold.

17. The method of claim 16, in which the reinforcement element comprises a metal wire configured in a spiral pattern around an inner surface of the conduit.

18. The method of claim 17, in which determining the first physical property comprises determining strain in the reinforcement element using a strain gage.

19. The method of claim 16, in which the conduit conducts hydraulic fluid on a machine, and in which determining the fault condition is performed by an electronic control module provided on the machine.

20. The system of claim 19, in which the electronic control module is operably coupled to an engine, and in which the electronic control module is further configured to communicate an override signal to the engine in response to the fault condition.
